(19) United States
(12) Patent Application Publication
(10) Pub. No.: US 2026/0207182 A1
(43) Pub. Date: Jul. 23, 2026

(54) Positioning Needle

(71) Applicant: Stichting Radboud universitair medisch centrum, Nijmegen (NL)

(72) Inventor: Dennis George Hugo Bosboom, Nijmegen (NL)

(73) Assignee: Stichting Radboud universitair medisch centrum, Nijmegen (NL)

(21) Appl. No.: 19/139,586

(22) PCT Filed: Dec. 14, 2023
(86) PCT No.: PCT/EP2023/085910

§ 371 Date: Jun. 16, 2025

(30) Foreign Application Priority Data
Dec. 19, 2022 (EP) 22214661.5

(51) Int. Cl. A61B 10/02; A61B 10/04

(52) U.S. Cl. CPC A61B 10/0233; A61B 2010/045

(57) ABSTRACT

A puncture assembly for imaging-guided medical procedures to reach a target in deep tissues. The puncture assembly comprising, a tubular member having a wall enclosing a lumen and comprising a distal section extending up to a distal end of the tubular member, wherein the distal section is open on one side up to and including the distal end of the tubular member; and a core, slidably and rotatably disposed in the lumen of the tubular member and having a distal section, wherein the distal section is flexible and biased towards a curved shape.

### FIELD OF THE INVENTION

The invention relates to a puncture assembly and a method for guiding a tubular member towards a target tissue.

### BACKGROUND OF THE INVENTION

Minimally invasive image-guided medical procedures may be performed with the aid of a puncture instrument or needle in a body with the goal to reach one or more specific targets located in deep tissues. It is often the case that a target is missed at first insertion, resulting in a position of the tip of the needle nearby the target region. To correct this, the needle may be maneuvered inside the body. Alternatively, a re-insertion of the needle or insertion of a new needle, may be performed. Both approaches result in further harm to tissues being done.

A target may be missed, for example, because the medical image provides limited information on the exact location of the target. Another reason is because the target composition usually differs in relation to the surrounding tissues, the target region may slip or drift away from the needle when the needle is introduced.

An alternative approach to prevent slippage of the target is first inserting another needle to stabilize the neighboring target tissues, such that the target can be reached. Nevertheless, this alternative requires at least two needles and at least two needle insertions.

CN 103257053 A describes a self-deformable multi-axial puncture device composed of a deformable puncture needle cannula made by a shape memory alloy and an arc-shaped puncture needle core. However, this solution still involves the risk of missing the target. In general, there is the need to for an improved puncture assembly.

### SUMMARY OF INVENTION

It would be advantageous to provide an improved puncture assembly.

Therefore, according to a first aspect of the invention, there is provided a puncture assembly, comprising:
a tubular member having a wall enclosing a lumen and comprising a distal section extending up to a distal end of the tubular member, wherein the distal section of the tubular member is open on one side up to and including the distal end of the tubular member and closed on another side up to and including the distal end of the tubular member; and
a core slidably and rotatably disposed in the lumen of the tubular member and having a distal section, wherein the distal section is flexible and biased towards a curved shape.

The puncture assembly allows to reach one or more targets more efficiently, which may reduce harm done to tissues. The puncture assembly allows to fixate the target area while aiming at the target area. In case a target is missed, the target may be fixated by virtue of the tubular member present nearby the target. The core may, by virtue of its curved shape and the open side of the distal section of the tubular member, reach out from the tubular member laterally. By virtue of the open side of the distal section of the tubular member extending up to and including the distal end of the tubular member, it is possible to partially retract the tubular member while the core extends laterally through the open side. Moreover, it is possible to thereafter rotate the tubular member with respect to the core, so that the closed side of the distal section aligns with the bending direction of the biased core. After that the tubular member may be re-inserted, so that it may be guided by the core towards the target.

If, during a medical procedure, the tubular member reaches the target successfully, the procedure can be continued according to the medical protocol. Thus, if the first puncture is successful the core does not have to be used.

The distal section of the tubular member, open on one side up to and including the distal end of the tubular member, may provide cutting means to penetrate the surface tissues of a body more easily and extend towards a specific target in deep tissues.

The distal section of the tubular member may have a length (L1), and the distal section of the core may be biased towards a curve with a radius (R), wherein the length (L1) divided by the radius (R) is at least 0.2, preferably at least 0.4. The length of the distal section of the tubular section facilitates the fixation of neighboring (target) tissues.

In typical implementations, the distal section of the tubular member may have a length between 5 mm and 30 mm, preferably between 15 mm and 30 mm. This makes the puncture assembly particularly useful for common medical applications and interventions, such as biopsies.

The distal section of the core may have a biased curvature with a radius between 5 mm and 60 mm. This makes the puncture assembly particularly useful for common medical applications, such as biopsies.

The tubular member may have a beveled tip. This has the advantage to provide additional sharpness and to facilitate the puncture and penetration of the tubular member into the tissues of the body. Similarly, the core may have a beveled tip, which can provide cutting means to facilitate the puncture of the target.

The core may be hollow. A hollow construction provides the core with a channel that can be used for various applications such as inoculating or extracting a fluid or providing a further component.

The core may be longer than the tubular member, which has the advantage of facilitating the manipulation of the core from the proximal end of the core.

The puncture assembly may comprise an indicator to indicate the axial and/or radial position of the core relative to the tubular member. For example, the radial position may be indicative of a rotation along a longitudinal axis of the core relative to the tubular member. This has the advantage to facilitate to the user the manipulation of the puncture assembly. For example, it helps to align the open and closed side of the distal section with the bias of the distal section of the core. The indicator may be located at a proximal portion of the tubular member or a proximal portion of the core. The indicator may be located on a handpiece at a proximal end of tubular member and/or core.

A cross section of the distal section of the tubular member may have a semicircular shape, which has the advantage of improved guidance of the tubular member by the core. The semicircular shape may correspond to, for example, between 25% and 65% of the circumference of the closed portion of the tubular member, preferably between 45% and 55% thereof. This helps to guide the tubular member over the core.

The cross section may be the same along the distal section. Alternatively, the shape of the cross section may vary over the length of the distal section, which may improve the cutting properties at the distal end of the tubular member and/or may provide the distal end of the core, when in use, radial freedom as it is being extended from the distal section of the tubular member. For example, the percentage of the circumference of the tubular member covered by the cross section of the closed side of the distal section may distally decrease from at least 55% at a proximal end of the distal section to at most 25% at the distal end of the distal section. Said structure may provide an effective sharpness.

A transition at a proximal end of the distal section, between the distal section and a closed portion of the tubular member, may have a rounded shape, which may have the advantage to facilitate the penetration of the distal portion of the tubular member into the deep tissues of the body.

According to a second aspect of the invention, and in accordance with the advantages and effects described herein above, there is provided a method to guide a tubular member towards a target, the method comprising:
providing the tubular member near the target, the tubular member having a wall enclosing a lumen and comprising a distal section extending up to a distal end of the tubular member, wherein the distal section is open on one side up to and including the distal end of the tubular member and closed on another side up to and including the distal end of the tubular member. The tubular element may be provided near the target with the open side of distal section facing any radial direction relative to the target, in this case, the tubular element may be rotated until the open side of the distal section faces towards the target. Preferably, the tubular element is provided with the open side of the distal section facing towards the target.
providing a core in the lumen of the tubular member, the core having a distal section, wherein the distal section is flexible and biased to a curved shape, such that the biased distal section is biased towards the open side of the distal section. The core may be provided before or after the tubular element is provided near the target. The core may be provided in the lumen with its curved distal section pointing to any radial direction relative to the target, in this case, the core maybe rotated until the curved distal section points towards the target. Preferably, the core is provided together with the tubular element near the target, and with its distal end aligned to the proximal part of the distal section of the tubular element, and with its curved distal section pointing towards the target
sliding the core in the lumen of the tubular member until its distal section extends from the distal section of the tubular member according to its curved shape;
retracting the tubular member;
rotating the tubular member around the longitudinal axis so that the closed side of the distal section of the tubular member is on the side of the curved distal section of the core; and
sliding the distal end of the tubular member over the core in distal direction.

The person skilled in the art will understand that the features described above may be combined in any way deemed useful. Moreover, modifications and variations described in respect of the puncture assembly may likewise be applied to the method, and modifications and variations described in respect of the method may likewise be applied to the puncture assembly.

### BRIEF DESCRIPTION OF DRAWINGS

In the following, aspects of the invention will be elucidated by means of examples, with reference to the drawings. The drawings are diagrammatic and may not be drawn to scale. Throughout the drawings, comparable items may be marked with the same reference numerals.

FIG. 1 shows a perspective view of a puncture assembly according to the present disclosure.

FIG. 2 shows a perspective view of a distal portion of the tubular member of the puncture assembly of FIG. 1.

FIG. 3 shows a perspective view of the distal portion of the core of the puncture assembly of FIG. 1.

FIG. 4A shows a longitudinal cross section of the puncture assembly of FIG. 1 with the core in a retracted position.

FIG. 4B shows a longitudinal cross section of the puncture assembly of FIG. 1, with the core in an extended position.

FIGS. 5A and 5B illustrate, correspondingly, geometric parameters of the tubular member and the core of FIG. 1.

FIG. 6A shows a radial cross section of the tubular member of FIG. 2.

FIG. 6B shows a radial cross section of a tubular member of a puncture assembly according to an alternative configuration.

FIGS. 7A-D show side views of different configurations of the distal section of the tubular member.

FIG. 8 shows an example of indicators at a distal portion of the tubular member and the core.

FIG. 9 illustrates a puncture assembly having a visual indicator at a proximal portion.

FIG. 10A-H illustrate aspects of a method of guiding a tubular member towards a target.

### DETAILED DESCRIPTION OF EMBODIMENTS

Certain exemplary embodiments will be described in greater detail, with reference to the accompanying drawings. The matters disclosed in the description, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail since they would obscure the description with unnecessary detail. Certain embodiments of the present disclosure can be used during medical procedures such as biopsy, drainage, surgery, injection, and ablation. Yet, the present invention is suitable for any procedure that involves reaching one or more targets.

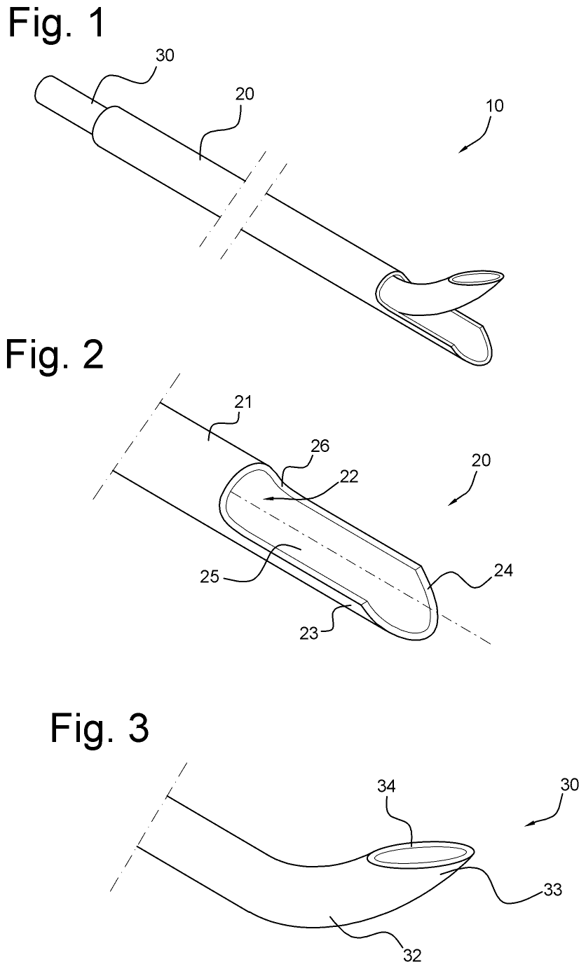

FIG. 1 shows a perspective view of a puncture assembly 10 according to the present disclosure. The puncture assembly 10 comprises a tubular member 20 and a core 30.

FIG. 2 shows a perspective view of a distal portion of the tubular member 20 of FIG. 1. The tubular member 20 has a tubular body comprising a wall 21 that encloses a lumen 22. A distal section 25 extending up to a distal end 23 is open on one side up to and including the distal end 23 of the tubular member 20. In the presented embodiment, the tubular member 10 has a beveled tip 24. The tubular member 20 may be used alone or together with the core during a medical procedure.

In certain embodiments, the tubular member 20 may be substantially stiff or slightly flexible or may have different degrees of flexibility over its length, for example, being its distal section 25 less stiff than a proximal portion. For example, the tubular member 20 is not biased to a curved shape to effectively penetrate superficial and deep tissues. Moreover, the tubular member 20 may have substantially the shape of a straight tube, e.g., to easily direct the distal section 25 towards a target. For example, the wall thickness of the tubular member may be about 0.3 mm. In certain embodiments, such as biopsy device, the tubular member 20 may have an outer diameter of at most 3 mm. For example, the inner diameter of the lumen of the tubular member may be at most 2.4 mm in these certain embodiments such as biopsy device. In an alternative embodiment, the puncture assembly could be e.g., a surgical device or catheter, wherein the tubular member may have an outer diameter of between, for example, 5 mm and 15 mm, preferably between 8 mm and 12 mm. For example, the wall thickness could be in a range of 0.2 mm to 2 mm in such embodiments.

FIG. 3 shows a perspective view of the distal portion of the core of FIG. 1. In the exemplary embodiment, the core 30 has a slightly flexible elongated body with a flexible distal section 32 that is biased towards a unidirectional curved shape (i.e., the curve extends in a plane). Preferably, the rest of the body of the core 30 remains unbiased. The elongated body of the core 30 may be solid or hollow. A hollow construction, as shown in FIG. 3, may provide the core with a lumen. Such a lumen may be used for various purposes, such as transport of fluids or providing another component through the lumen. In certain embodiments, the distal section 32 of the core 30 may be biased in more than one direction over its length, which can facilitate reaching targets in a more complex situation. In the presented embodiment, the core 30 has a distal end 33 with a beveled tip 34.

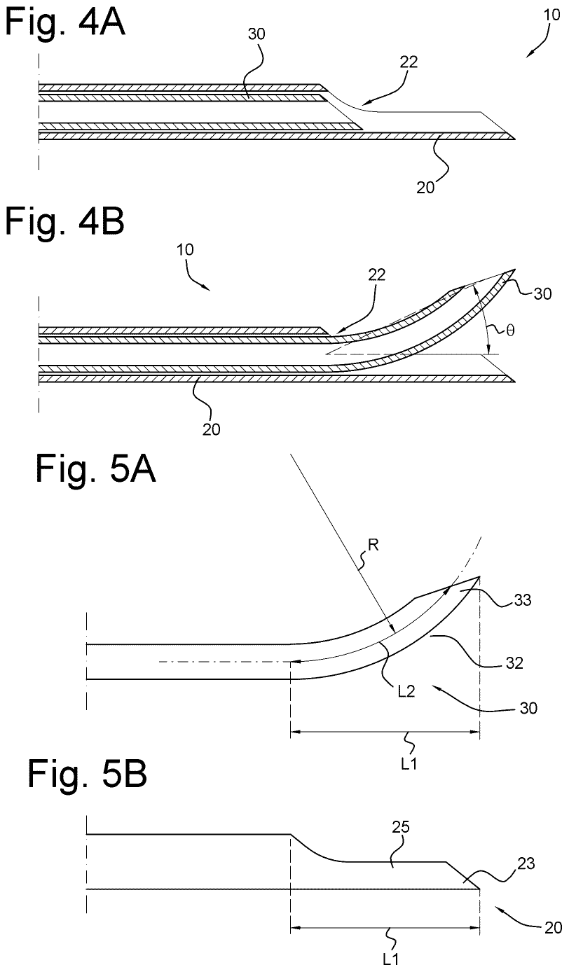

The distal section 32 of the core may be flexible such that it can adopt an (almost) straight shape and be disposed in the lumen 22 of the tubular member 20, as shown in FIG. 4A. In this example, the distal section 32 of the core 30 may be disposed in the lumen 22 of the tubular member 20, proximal to the distal section 25 of the tubular member 20. In certain embodiments, the distal section 32 may have a distal end 33 (wherein the distal end 33 may end at the distal tip of the core 30), which distal end 33 may be more rigid than the distal section 32, which may enhance the puncturing properties of the core 30. For example, this may make it easier to use the core 30 to penetrate tissues to reach multiple aimed targets located in the vicinity of the distal section 25 of the tubular member 20.

In FIG. 4A, a longitudinal cross-section of the puncture assembly of FIG. 1 shows the core 30 in a retracted position inside the lumen 22 of the tubular member 20. Thus, the flexibility of the distal section 32 of the core 30 facilitates its manipulation, radially and axially, inside the lumen 22 of the tubular member 10. FIG. 4B shows the distal section 32 of the core 30 in a position extending from the lumen 22 of the tubular member 20. The core 30 can be used in case that, for example, a target is located in the vicinity of the distal section 25 of the tubular member 20 or for when multiple targets are aimed sequentially. The distal end 33 of the core 30 may be at an angle theta (θ) to the longitudinal axis of the core, where θ may be between 15 and 90 degrees, preferably, between 25 and 60 degrees. The flexibility of the core 30 and its distal section 32 may be adapted according to the axial stiffness and shape of the tubular member 10. That is, in general the stiffness of the wall 21 of the tubular member 20 is greater than the stiffness of the distal section 32 of the core 30. In certain embodiments, the core 30 may be entirely biased to a curved shape, with a flexibility that allows it to be radially and axially manipulated inside the lumen 22 of the tubular member 20.

The outer diameter of the core 30 may be smaller than the inner diameter of the tubular member 20 (or the diameter of the lumen 22), to easily slide and rotate the core 30 with respect to the tubular member 20. In certain embodiments, where the puncturing assembly is for e.g., biopsy, the core 30 may have an outer diameter of at most 2.7 mm, preferably at most 2.4 mm, and at least 1 mm. In an alternative embodiment, when the puncturing assembly is for example a catheter or surgical device, the core 30 may have an outer diameter between 3 mm and 10 mm, preferably between 6 mm and 10 mm. It will be understood that the inner and outer diameters of the tubular member 20 and the outer diameter of the core 30 can depend on their material properties and shape, thus the difference between diameters may be adapted accordingly.

FIGS. 5A and 5B illustrate, correspondingly, geometric parameters of the tubular member and the core of FIG. 1. The length L1 of the distal section 25 of the tubular member 10 may control how much tissue will be fixated thereby. The curvature R of the biased shape of the distal section 32 of the core 30, as well as the length L2 of the distal section 32 of the core, may control the lateral distance that may be reached from the tubular element 20 by the tip 34 of the core 30.

In the case that the core 30 is used during a medical procedure, the reachable space of its distal section 32 is defined by its curvature R and length L2. There is an interaction between L1, L2, and R that contributes to the effectiveness of the puncture assembly 10 to reach a target. For these reasons, in certain embodiments, the ratio L1/R between the length L1 of the distal section 25 of the tubular member 20 and the curvature R of the distal section 32 of the core 30, may be at least 0.2, preferably at least 0.4, more preferably at least 0.8. Moreover, this ratio may be preferably at most 1.2. Moreover, the length L2 of the distal section 32 of the core 30 may be at least L1/2, preferably at least equal to L1. As in certain embodiments, the entire core 30 is biased, in principle there is no upper limit on the length L2 of the distal section 32 of the core 30.

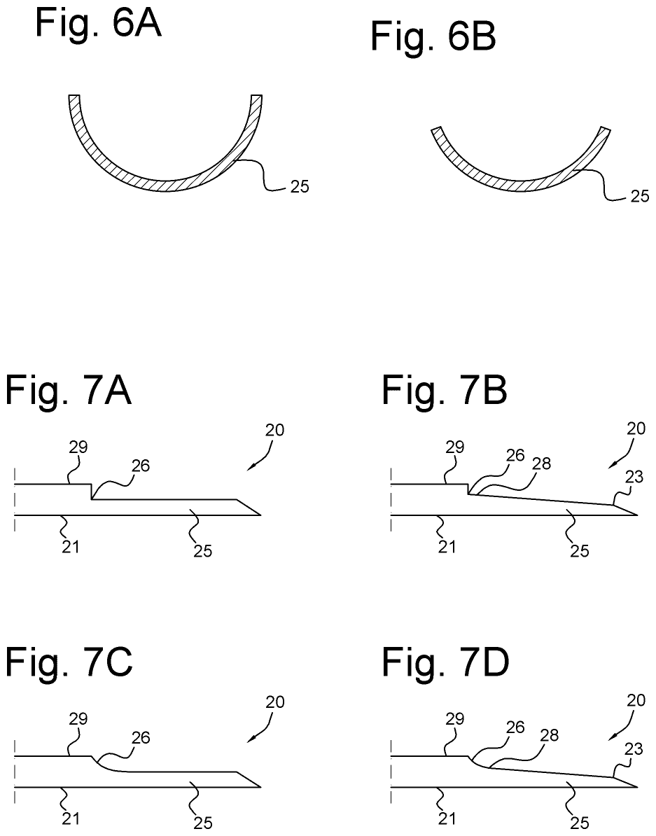

FIG. 6A shows a cross section of the distal section 25 of the tubular member 20 of FIG. 2. In certain embodiments, the distal section 25 of the tubular member may have a semicircular cross-section, as shown in FIG. 6A-B. Preferably, the semicircular may correspond to a range between 25% and 65% of the circumference of the wall 21 of the tubular member 20, more preferably between 45% and 55%. This range, first steers radially the distal section 32 of the core 30 towards a direction perpendicular to the longitudinal axis of the tubular member 20, facilitating reaching the target by the core 30; and second, guides the tubular member 20 over the core 30, facilitating reaching the target by the distal section 25 of tubular element 20.

The shape of the distal section 25 of the tubular member 20 may be constant or vary over its length L1, e.g., tapering towards the distal end, as in certain embodiments illustrated in FIGS. 7A-D that show side views of different configurations of the distal section 25 of the tubular member 20. In FIGS. 7B and 7D, for example, the percentage of the circumference of the wall 21 of the tubular member 10 covered by the semicircular decreases from at least 55% at a proximal end 28 of the distal section 25 to at most 25% at the distal end 23 of the distal section 25.

In certain embodiments, a transition 26, between the distal section 25 and a closed portion 29 of the tubular member 20 may be rounded as exemplified in FIGS. 7C and 7D, or may have an angular shape, as exemplified in FIGS. 7A and 7B. A rounded shape may be preferred because it facilitates the penetration of the tubular member into the deep tissues of the body.

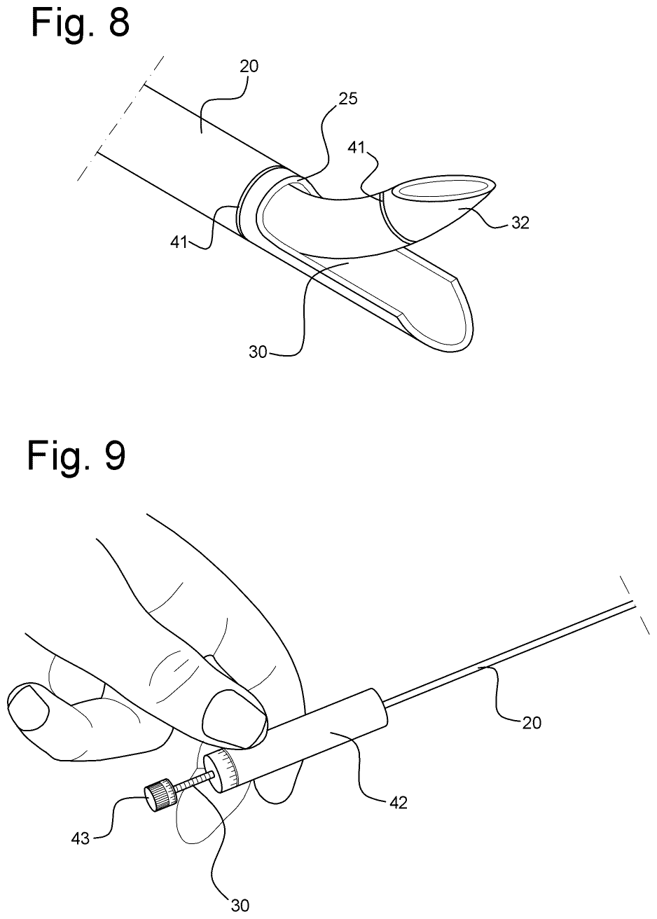

The tubular member 20 and the core 30 may comprise an indicator to indicate to the user the axial and/or radial position relative to each other. This has the advantage to facilitate to the user the manipulation of the puncture assembly and specifically provide a more precise axial and/or radial position of the distal section 32 of the core 30 relative to the tubular member 20. In certain embodiments, as shown in FIG. 8, indicators 41 with a contrasting property, such as color, perceptible with the imaging devices, may be indirectly visualized by being located adjacent to the distal portion 25 of the tubular member 20 and/or the core 30.

In certain embodiments, the indicators may be directly visualized by being located at the proximal portion of the tubular member and/or the core. Radial indicators at the proximal portions may be printed or engraved markers or graduated/tapered circular markers. For example, FIG. 9 shows the proximal portion of the puncture assembly, the tubular member 20 and core 30, respectively comprise on their surface hubs 42 and 43, which circumference is graduated to indicate rotation around the longitudinal axis. Indicators in other embodiments at the proximal portion of the puncture assembly may be printed or engraved longitudinal indicators and may extend over the surface of the core, which may be graduated and to provide the user information regarding the axial position of the distal section of the core relative to the tubular member. In other implementations, the tubular member 20 and the core 30 may be operated by actuators located at the proximal portion of the puncture assembly. The actuators may be controlled by a computer that is configured to plan the axial and radial trajectories of the tubular member 20 and the core 30 depending on the position of the target, or targets in the body.

The tubular member 20 of the present invention can be inserted to provide access to deep or superficial tissues under the skin. The open side of the distal section 25 of the tubular member 20 has the advantage to provide different functionalities, for example:
First, it provides puncture properties to penetrate through deep tissues in the body that may have different mechanical properties (i.e., rigidity, viscosity, etc.). Secondly, after being inserted, it provides mechanical stability to the tissues surrounding its distal end 23, facilitating the reach of a target 60 located in its the vicinity. Thirdly, it provides the core 30 a distal and lateral passage, which facilitates the reach of targets that are located radially from the distal section 25 of the tubular member 20.

The distal section 32 of the core 30 biased to a curve shape has the advantage of reaching targets located in the vicinity of the distal section 25 of the tubular member 20. Its flexibility allows it to accommodate to the axial shape of the lumen 22 of the tubular member 20 and it can directly retain the target 60 and secure its position while the tubular member 20 is guided towards it.

The distal section 25 of the tubular member 20 may be guided towards the target 60 by following the path created by the core 30. The fact that the tubular element 20 is rotatable, such that the open side of its distal section 25 faces towards a direction opposite to the curved shape, has a first advantage of facilitating the tubular member 20 to reach the target 60 with its most distal end 23, hence minimizing the amount of extension necessary to get to the target 60. A second advantage is that the distal section 25 of the tubular member 20 straightens the distal section 32 of the core 30 as it is being extended towards the target 60, this prepares the components of the puncture assembly to be withdrawn together from the body, thus simplifying the procedure to the user.

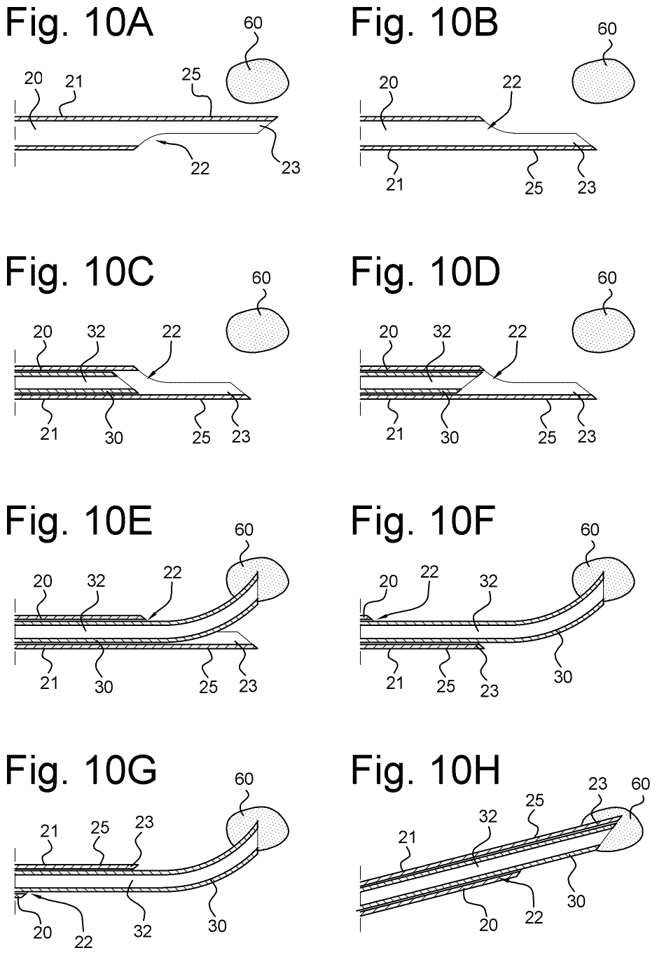

A second aspect of the present disclosure relates to a method to guide a tubular member towards a target, as exemplified by FIGS. 10A-H. This method can be used when a target is located nearby a tubular member or when multiple targets are pursued. That may be the case of, for example, minimally invasive medical procedures that begin puncturing the skin and inserting a tubular member in the body. The method may comprise:
inserting a tubular member 20 near a target 60, the tubular member 20 having a wall 21 enclosing a lumen 22 and comprising a distal section 25 extending up to a distal end 23 of the tubular member 20, wherein the distal section 25 is open on one side up to and including the distal end 23 of the tubular member 20. The tubular member 10 may be inserted with the open side of the distal section 25 facing opposite (see FIG. 10A), towards (see FIG. 10B), or other direction to the target. Preferably the tubular member 20 is inserted in a radial orientation such that its open distal section 25 is facing towards the target 60. Alternatively, the tubular member 20 may be rotated around the longitudinal axis such that the open side of the distal section 25 faces towards the target 60. The open side of the distal section 25 of the tubular member 20 has the advantage to provide different functionalities, for example: First, it provides puncture properties to penetrate through deep tissues in the body that may have different mechanical properties (i.e., rigidity, viscosity, etc.). Secondly, after being inserted, it provides mechanical stability to the tissues surrounding its distal end 23, facilitating the reach of a target located in its the vicinity by means of the core 30. Thirdly, it provides a distal and lateral passage, which facilitates the reach of targets that are located radially from its distal section 25.
providing a core 30 in the lumen 22 of the tubular member 20, the core 30 having a distal section 32, wherein the distal section 32 is flexible and biased to a curved shape. The core 30 may be provided in the lumen 22 before or after the insertion of the tubular member 20 in the body, depending on the procedure. In said first situation, the distal section 32 of the core 30 maybe aligned or adjacent proximally to the distal section 25 of the tubular member 20 before insertion. In any situation, the core 30 may be provided in a radial position such that its biased distal section 32 is pointing opposite (see FIG. 10C), towards (see FIG. 10D), or other direction to the target. Preferably the core 30 is provided before inserting the tubular member with its biased distal section 32 pointing towards the open side of the distal section 25. In that case the core 30 may be provided inside and up to a distal end 23 of the closed portion of the tubular member 20, not extending into the distal section 32. Alternatively, the core 30 may be rotated around the longitudinal axis after the step of inserting the tubular member 20, and with the core 30 inside the closed portion 29 of the tubular member 20, substantially not extending into the distal section 25 of the tubular member 20, such that its curved distal section 32 is biased towards the open side of the distal section 25. After inserting the tubular member 20, the target 60 may be in the vicinity of its distal section 25. The distal section 32 of the core 30 biased to a curve shape has the advantage of reaching targets located in the vicinity of the distal section of the tubular member.
sliding the core 30 in the lumen 22 of the tubular member 20 in distal direction until its distal section 32 extends from the distal section 25 of the tubular member 20 according to its curved shape (see FIG. 10E), up until the target 60 is reached. Once the target is reached, depending on the procedure, the distal end 32 of the core 30 may optionally pierce, and/or transport fluids to, the target 60. The distal end 32 of the core 30 can, by its mere presence, mechanically stabilize the surrounding tissues including the target 60. Depending on the procedure, for example, when multiple targets are aimed, the core 30 may be retracted to an extend such that its distal section 32 adopts an (almost) straight shape in the lumen 22 of the tubular member 20. Consecutively, the core 30 may be rotated around its longitudinal axis and then extended again up until the next target is reached. The steps of retracting, rotating, and extending the core 30 may be repeated sequentially until all aimed targets have been reached.
retracting the tubular member 20. Once the target is reached by the core 30, the distal section 25 of the tubular member 20 may be guided towards the target 60 by first retracting the tubular member 20; the amount of retraction can be such that the distal section 25 aligns to the core 30 (see FIG. 10F).
rotating the tubular member 20 around the longitudinal axis by for example around 180 degrees, so that the curved distal section 32 of the core 30 points towards the closed side (in the semicircular shape) of the distal section 25, i.e., the open side of the distal section 25 faces to a direction opposite to the curved shape (see FIG. 10G). The rotation of the tubular member 20 ensures that its distal section 25 will reach the target 60 while partially influencing the shape distal section 32 of the core 30 to a straighter shape.
sliding the distal section 25 of the tubular member 20 over the core 30 in distal direction. The tubular member 20 can be slid in distal direction to effectively reach the target 60, guided by the core 30 (see FIG. 10H). After that, if desired, the core 30 may be removed altogether, leaving the tubular member 20 in place with its distal tip at the target position.

The step of retracting the tubular member 20 and all following steps are optional. In alternative embodiments, the step of retracting the tubular member 20 comprises removing the tubular member 20, and the two steps of the method after that may be omitted. After that, any other component may be advanced towards the target using the core 30 as a guide. Yet alternatively, a fluid or other material may be brought to the target 60 through the optional lumen of the core 30.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims, or the description may be implemented as a single hardware item combining the features of the items described.

Certain aspects are defined in the following clauses.
1. A puncture assembly (10) comprising:
   a tubular member (20) having a wall (21) enclosing a lumen (22) and comprising a distal section (25) extending up to a distal end (23) of the tubular member (20), wherein the distal section (25) of the tubular member (20) is open on one side up to and including the distal end (23) of the tubular member (20) and closed on another side up to and including the distal end (23) of the tubular member (20); and
   a core (30), slidably and rotatably disposed in the lumen (22) of the tubular member (20) and having a distal section (32), wherein the distal section (32) is flexible and biased towards a curved shape.
2. The puncture assembly of clause 1, wherein the distal section (25) of the tubular member (20) has a length (L1) and the distal section (32) of the core (30) is biased towards a curve with a radius (R), wherein the length (L1) divided by the radius (R) is at least 0.2, preferably at least 0.4.
3. The puncture assembly of clause 1, wherein the distal section (25) of the tubular member (20) has a length (L1) between about 5 mm and 30 mm, preferably between 15 mm and 30 mm.
4. The puncture assembly of any of the preceding clauses, wherein the distal section (32) of the core (30) has a biased curvature with a radius (R) between 5 mm and 60 mm.
5. The puncture assembly of any of the preceding clauses, wherein the tubular member (20) has a beveled tip.
6. The puncture assembly of any of the preceding clauses, wherein the core (30) has a beveled tip.
7. The puncture assembly of any of the preceding clauses, wherein the core (30) is hollow.
8. The puncture assembly of any of the preceding clauses, wherein the wall (21) of the tubular member is stiff.
9. The puncture assembly of any of the preceding clauses, further comprising an indicator to indicate axial position and/or radial position relative to each other.
10. The puncture assembly of clause 9, wherein the indicator is located at a proximal portion (24) of the tubular member (20) or a proximal portion (31) of the core (30).
11. The puncture assembly of any of the preceding clauses, wherein a cross-section of the closed side of the distal section (25) of the tubular member (20) has a semicircular shape.
12. The puncture assembly of clause 11, wherein the semicircular cross-section corresponds to between 25% and 65% of the circumference of the tubular member (20), preferably between 45% and 55%.
13. The puncture assembly of clause 11 or 12, wherein the semicircular cross-section varies over the length of the distal section (25).
14. The puncture assembly of clause 12, wherein the percentage of the circumference of the tubular member (20) covered by the semicircular cross-section distally decreases from at least 55% at a proximal end of the distal section (25) to at most 25% at a distal end of the distal section (25).
15. The puncture assembly of any one of the preceding clauses, wherein a transition (26) at a proximal end of the distal section (25), between the distal section (25) and a closed portion of the tubular member (20), is rounded.
16. A method for guiding a tubular member towards a target, the method comprising:
   a) providing the tubular member (20) near the target, the tubular member (20) having a wall (21) enclosing a lumen (22) and comprising a distal section (25) extending up to a distal end (23) of the tubular member (20), wherein the distal section (25) is open on one side up to and including the distal end of the tubular member (20) and closed on another side up to and including the distal end of the tubular member (20);
   b) providing a core (30) in the lumen (22) of the tubular member (20), the core (30) having a distal section (32), wherein the distal section (32) is flexible and biased to a curved shape, such that the biased distal section (32) is biased towards the open side of the distal section (25); and
   c) sliding the core (30) in the lumen (22) of the tubular member (20) until its distal section (32) extends from the distal section (25) of the tubular member (20) according to its curved shape.
17. The method of clause 16, further comprising:
   d) retracting the tubular member (20);
   e) rotating the tubular member (20) around the longitudinal axis so that the closed side of the distal section (25) of the tubular member (20) is on the side of the curved distal section (32) of the core (30); and
   f) sliding the tubular member (20) over the core in distal direction.
18. The method of clause 16 or 17, wherein step a) of providing the tubular element (20) near the target (60) further comprises:
   rotating the tubular element (20) around the longitudinal axis such that its open distal section (25) faces towards the target (60).
19. The method of any one of clauses 16 to 18, wherein step b) of providing a core (30) in the lumen (22) of the tubular member (20), further comprises:
   rotating the core (30) around the longitudinal axis such that its curved distal section (32) points towards the open side of the distal section (25).

1. A puncture assembly comprising:
a tubular member having a wall enclosing a lumen and comprising a distal section extending up to a distal end of the tubular member, wherein the distal section of the tubular member is open on one side up to and including the distal end of the tubular member and closed on another side up to and including the distal end of the tubular member; and
a core slidably and rotatably disposed in the lumen of the tubular member and having a distal section, wherein the distal section is flexible and biased towards a curved shape, wherein the core is configured to reach out from the tubular member laterally.

2. The puncture assembly of claim 1, wherein the distal section of the tubular member has a length and the distal section of the core is biased towards a curve with a radius wherein the length divided by the radius (R) is at least 0.2, preferably at least 0.4.

3. The puncture assembly of claim 1, wherein the distal section of the tubular member has a length between about 5 mm and 30 mm, preferably between 15 mm and 30 mm.

4. The puncture assembly of claim 1, wherein the distal section of the core has a biased curvature with a radius between 5 mm and 60 mm.

5. The puncture assembly of claim 1, wherein one or both of the tubular member and the core has a beveled tip.

6. The puncture assembly of claim 1, wherein the core is hollow.

7. The puncture assembly of claim 1, wherein the wall of the tubular member is stiff.

8. The puncture assembly of claim 1, further comprising an indicator to indicate axial position and/or radial position relative to each other.

9. The puncture assembly of claim 8, wherein the indicator is located at a proximal portion of the tubular member or a proximal portion of the core.

10. The puncture assembly of claim 1, wherein a cross-section of the closed side of the distal section of the tubular member has a semicircular shape.

11. The puncture assembly of claim 10, wherein the semicircular cross-section corresponds to between 25% and 65% of the circumference of the tubular member, preferably between 45% and 55%.

12. The puncture assembly of claim 10, wherein the semicircular cross-section varies over the length of the distal section.

13. The puncture assembly of claim 10, wherein the percentage of the circumference of the tubular member covered by the semicircular cross-section distally decreases from at least 55% at a proximal end of the distal section to at most 25% at a distal end of the distal section.

14. The puncture assembly of claim 1, wherein a transition at a proximal end of the distal section between the distal section and a closed portion of the tubular member is rounded.

15. A method for guiding a tubular member towards a target, the method comprising:
providing the tubular member near the target, the tubular member having a wall enclosing a lumen and comprising a distal section extending up to a distal end of the tubular member wherein the distal section is open on one side up to and including the distal end of the tubular member and closed on another side up to and including the distal end of the tubular member
providing a core in the lumen of the tubular member, the core having a distal section, wherein the distal section is flexible and biased to a curved shape, such that the biased distal section is biased towards the open side of the distal section; and sliding the core in the lumen of the tubular member until its distal section extends from the distal section of the tubular member according to its curved shape, wherein the core extends from the tubular member laterally.
